(19) United States
(12) Patent Application Publication
(10) Pub. No.: US 2007/0059257 A1
(43) Pub. Date: Mar. 15, 2007

(54) DENTINAL COMPOSITION FOR HYPERSENSITIVE TEETH

(75) Inventors: David Estrada, Jersey City, NJ (US); Benjamin D. Fitz, Brooklyn, NY (US); Jan F. Macik, Clark, NJ (US); Neil J. Napolitano, Fanwood, NJ (US); Alfred J. Smetana, Wayne, NJ (US); Joseph D. Synodis, Summit, NJ (US); Michael E. Trama, River Vale, NJ (US)

Correspondence Address: GLAXOSMITHKLINE;Corporate Intellectual Property - UW 2220, P.O. Box 1539, King of Prussia, PA, 19406-0939, US

(73) Assignee: Block Drug Company, Inc.

(21) Appl. No.: 11/554,374

(22) Filed: Oct. 30, 2006

Related U.S. Application Data
(63) Continuation of application No. 10/344,074, filed on Feb. 6, 2003.
Application No. 10/344,074 is a continuation of application No. 09/850,352, filed on May 7, 2001, now abandoned.
Application No. 09/850,352 is a continuation-in-part of application No. 09/642,048, filed on Aug. 18, 2000, now Pat. No. 6,536,737.

(51) Int. Cl. A61K 8/21; A61K 8/46

(52) U.S. Cl. 424/52; 424/57

(57) ABSTRACT

A composition for the treatment of sensitive teeth comprising a suitable carrier and a desensitizing amount of: a) at least one tubule blocking agent preferably a combination of two such agents; and b) a nerve desensitizing agent selected from the group consisting of at least one potassium salt at least one strontium salt and mixtures thereof wherein the tubule blocking agent deposits or swells upon the dentinal surface and/or precipitates within the dentinal tubules retaining a higher concentration of the nerve desensitizing agent at the exposed dentinal surface and within the dentinal tubules The compositions may further contain a source of physiologically acceptable fluoride ion such as stannous fluoride Also disclosed are methods of using the subject compositions in the treatment of sensitive teeth.

### RELATED APPLICATIONS

This application claims the benefit of Ser. No. 09/850,352 filed May 7, 2001 which is a CIP of U.S. patent application Ser. No. 09/642,048 filed Aug. 21, 2000.

### BACKGROUND OF THE INVENTION

Dentinal hypersensitivity is a temporary induced pain sensation produced when hypersensitive teeth are subjected to changes in temperature and/or pressure or to chemical action. Hypersensitivity may occur whenever the dentin of a tooth is exposed by attrition or abrasion, or when the tooth's finer root surface is exposed by periodontal disease. Dentin is a bone-like material in teeth that is usually covered by enamel above the gum line and cementum below the gum line. The enamel or cementum may be removed through decay, injury, disease or other causes, thereby exposing the dentin to external stimuli in the mouth. Dentin generally contains channels, called tubules, that allow material and energy transport between the exterior of the dentin and the interior of the tooth where the nerve is located.

One theory of dentinal hypersensitivity, called the hydrodynamic theory, suggests that exposure of these tubules to external stimuli can cause irritation of the nerve and lead to the discomfort of hypersensitivity. The hydrodynamic theory suggests that hypersensitivity may be treated by making the nerve in the tooth less sensitive to stimuli, or by blocking or occluding the tubules to prevent or limit exposure of the nerve to external stimuli.

Many attempts have been made to control dentinal hypersensitivity. One approach is to reduce the excitability of the nerve in a sensitive tooth. This technique interferes with the ordinary triggering process of the nerve by altering the chemical environment of the nerve through the use of agents to make the nerve less sensitive. These agents are generally referred to as “nerve agents” or “nerve desensitizing agents”. The most well-known agent for this purpose is potassium nitrate, used in commercial dentifrices for sensitive teeth and discussed in U.S. Pat. No. 3,863,006. Examples of other agents known as nerve desensitizing agents are found in the following U.S. patents: potassium salts such as potassium bicarbonate and potassium chloride, U.S. Pat. Nos. 4,631,185 and 4,751,072; strontium and fluoride ions, U.S. Pat. No. 4,990,327: zinc and strontium ions, U.S. Pat. No. 3,888,976.

Another approach to control dentinal hypersensitivity, as discussed above with regard to the hydrodynamic theory, is the use of agents to fully or partially occlude tubules. Examples of such “tubule blocking agents” are found in the following patents: charged polystyrene beads, U.S. Pat. No. 5,211,939; apatite, U.S. Pat. Nos. 4,634,589 and 4,710,372;. a polyacrylic acid polymer having a typical molecular weight from about 450,000 to about 4,000,000, U.S. Pat. No. 5,270,031; and water-soluble or water-swellable polyelectrolytes or salts thereof, U.S. Pat. No. 4,362,713. In addition, U.S. Pat. No. 5,589,159 discloses the use of Laponite or hectorite clay to seal dentinal tubules.

PCT Patent Application WO 00042981 discloses a different mode of action wherein a composition of two different desensitizing agents (stannous fluoride and potassium nitrate) is separated in a dual phase dispensing unit. These agents are separated in the disclosed compositions to enhance their stability.

The inventors have surprisingly found a highly effective treatment of dentinal hypersensitivity in a composition that combines a nerve desensitizing agent with a tubule blocking agent, preferably a combination of two tubule blocking agents, thereby providing a synergistic effect over the prior art methods of employing either a nerve agent or a tubule blocking agent alone, or a nerve agent combined with a second chemical desensitizer.

### SUMMARY OF THE INVENTION

The invention provides a composition for desensitizing teeth comprising a desensitizing amount of: a) at least one tubule blocking agent, preferably a combination of two such agents; and b) a nerve desensitizing agent selected from the group consisting of at least one potassium salt, at least one strontium salt, and mixtures thereof, wherein the tubule blocking agent deposits or swells upon the dentinal surface and/or precipitates within the dentinal tubules, retaining a higher concentration of the nerve desensitizing agent at the exposed dentinal surface and within the dentinal tubules.

The invention further provides a method for desensitizing hypersensitive teeth by applying thereto a desensitizing amount of an oral composition comprising: a) a nerve desensitizing amount of at least one tubule blocking agent, preferably a combination of two such agents; and b) a desensitizing agent selected from the group consisting of consisting of at least one potassium salt, at least one strontium salt, and mixtures thereof.

This invention also discloses a method for preparing a composition for desensitizing teeth comprising a desensitizing amount of at least one tubule blocking agent, preferably a mixture of at least two tubule blocking agents, and a nerve desensitizing agent selected from the group consisting of at least one potassium salt, at least one strontium salt, and mixtures thereof.

### DETAILED DESCRIPTION OF THE INVENTION

The inventors have found a highly effective treatment of dentinal hypersensitivity in a composition that combines a tubule blocking agent with a nerve desensitizing agent, providing a synergistic and sustained effect over the prior art methods of employing either a nerve agent, a mixture of nerve agents, or a tubule blocking agent alone.

The first component of the subject is at least one tubule blocking agent, preferably a combination of two such agents as will be described hereinbelow, which serves to seal or block the dentin tubules thereby diminishing the effect of external stimuli such as changes in temperature, pressure, ionic gradients or contact with chemical irritants, as well as helping to reduce the flushing of potassium from the tubules by natural outward flow of dentinal fluid. Examples of materials which are used as tubule blocking agents include: materials having a particle size smaller than that of a dentin tubule for blocking the dentin tubules; and materials that can produce an agglomerate within the dentin tubules and/or form a precipitate that will deposit onto the dentinal surface.

Examples of tubule blocking agents useful in the compositions of the present invention include, without intended limitation, water absorbent, water-insoluble, acrylic polymers (cross-linked or otherwise); polymers, copolymers, fully or partially hydrolyzed polymers and copolymers, and full and partial salts thereof having repeating hydrophilic and hydrophobic groups within each molecule; hectorite clays including Laponite® clays; alkyl phosphate or fatty acid soaps, and mixtures thereof.

In one embodiment of the present invention, super-absorbent acrylic polymers having a particle size smaller than that of a dentin tubule are used as tubule blocking agents. Super-absorbent acrylic polymers are substantially water-insoluble, cross-linked, partially neutralized polymers that have been typically used in diapers, feminine hygiene articles and surgical dressings. They generally have a gel capacity of at least about 50 grams of deionized water per gram at 20° C. and often at least about 100 grams, 200 grams or more of water per gram dry weight of polymer at 20° C. Super-absorbent polymers may be a homopolymer of acrylic acid, or may be formed from a water-soluble blend of monoethylenically unsaturated monomers selected in known manner such that the final cross-linked polymer is water-absorbent. Such monomer blends may be nonionic, anionic, or cationic. Suitable cationic monomers that can be employed include acrylamides and dialkylaminoalkylmethacrylates, generally in the form of acid addition or quaternary ammonium salts. Nonionic monomers that can be employed include methacrylamide and the like.

Super-absorbent polymers may be, for example, a homopolymer of acrylic acid or may be formed from a water-soluble blend of monoethylenically unsaturated monomers selected in known manner so that the final cross-linked polymer is water-absorbent. Such monomer blends may be nonionic, anionic, or cationic. Suitable cationic monomers that can be employed include acrylamides and dialkylaminoalkylmethacrylates, generally in the form of acid addition or quaternary ammonium salts. Nonionic monomers that can be employed include methacrylamide and the like. Anionic monomers that can be employed include acrylic acid and the like. Preferred super-absorbing polymers for use as tubule blocking agents are cross-linked emulsion polymers of acrylic acid and copolymers of acrylic acid with acrylamide.

The particle size of the super-absorbent polymers utilized as tubule blocking agents is typically from about 0.5 to about 2.0 microns at their largest dimension before water absorption. Since the particles are irregular in shape, it will be understood that, so long as the particles are effective as tubule blocking agents, the size will be appropriate to the practice of the invention.

In another embodiment, acrylic polymers that can agglomerate within or over the dentin tubules are used as tubule blocking agents. Such polymers of polyacrylic acids or salts thereof are commonly used as binders in pharmaceutical and cosmetic formulations. Carbomers are a group of acrylic polymers that exhibit excellent general gelation properties and can act as mucoadhesives and counterirritants in formulations. They are commercially available under the trade name of Carbopol® from B F Goodrich. A preferred commercially available polymer for use as a tubule blocking agent is a polyacrylate polymer sold by B F Goodrich under the trade designation Carbopol® 980. It has been reported that certain Carbopol® polymers at concentrations of about 4% and above are effective against hypersensitivity. However, at this high concentration, and due to the thick mucilaginous nature of the gel formed upon application, the organoleptics of such polymers are not favorable.

In another embodiment, the tubule blocking agents are polymers having closely associated hydrophilic and hydrophobic regions. As used herein, “polymer” is intended to encompass both homopolymers and copolymers. Copolymers may include alternating copolymers, random copolymers, statistical copolymers, graft copolymers and block copolymers. Preferably, the tubule blocking agent is a homopolymer or an alternating copolymer, and more preferably an alternating copolymer. The copolymer may be linear, or branched, or even crosslinked to form a network polymer. More preferably, the copolymer has at least one hydrophilic monomer capable of forming a salt, and most preferably, the copolymer comprises a monovalent cation salt of a hydrophobic/hydrophilic copolymer that disperses into micelles in aqueous systems.

The hydrophobic monomer of the tubule blocking agent in this embodiment is preferably a long chain α-olefin while the hydrophilic monomer is preferably a strongly hydrophilic monomer that causes its associated copolymer form to create micelles. Preferred hydrophilic monomers are highly hydrophilic, salt-forming monomers, such as carboxylic acids and diacids. Most highly preferred hydrophilic monomers are diacids, such as maleic acid. Other anionic polymeric salts may also be used. Preferably, the copolymer is a regular copolymer that alternates between the two types of monomer in the copolymer chain.

The tubule blocking agent in the second embodiment is preferably a polysoap, i.e. the salt of a copolymer consisting of different, alternating monomeric sub units. One monomer is highly hydrophobic and preferably is a long chain α-olefin comprising a carbon chain of at least about eight (8), preferably at least about ten (10) and more preferably at least about twelve (12) carbon atoms. The second monomer is hydrophilic in character and preferably consists of a maleic acid or anhydride moiety. The tubule blocking agent in this second embodiment can be structurally represented as follows: 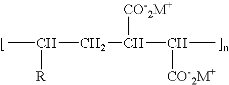
which is repeated any number of times (i.e., wherein n≧2) to produce the copolymer. R is a long chain aliphatic group comprised of a higher alkyl group, for example a hexyl group, i.e. the α-olefin monomer chain is C₈, or an octyl group, i.e. the α-olefin monomer chain is C₁₀, or longer group and M⁺ is a monovalent cation, preferably sodium, potassium, ammonium, choline, lysine, triethanolamine and mixtures thereof.

The hydrophobic/hydrophilic copolymer as a tubule blocking agent can comprise any number of repeating monomeric units. In general, the copolymer would have a molecular weight of from about 2,000 to about 1,000,000 daltons, preferably about 5,000 to about 500,000 daltons, and most preferably about 10,000 to about 100,000 daltons. Preferably, the copolymer is the hydrolysis product of an alternating long chain α-olefin-co-maleic anhydride copolymer wherein the α-olefin has at least about ten carbons and even more preferably at least about twelve carbons (e.g., C₁₂H₂₄) or higher. Examples of copolymers of this nature include but not limited to tetradecene/maleic anhydride copolymer, octadecene/maleic anhydride copolymer, triacontene/maleic anhydride copolymer, and mixtures thereof.

In one embodiment, the tubule blocking agent is Chevron/Gulf PA-18® brand polyanhydride resin, an alternating copolymer of a 1:1 molar ratio of maleic anhydride and 1-octadecene (CAS: “1-octadecene polymer with 2,5-furandione”; INCA: octadecene/MA copolymer). The resin has an average molecular weight from about 10,000 to about 60,000 daltons. Preferred for use as a tubule blocking agent is the hydrolyzed cation salt of this copolymer, especially the monovalent salts thereof. Preferred monovalent salts are the alkali salts and most preferably the sodium or potassium salts thereof and the monovalent cations of ammonium, triethanolamile, choline and lysine.

It has been found in accordance with the present invention that a combination of the previously described two groups of tubule blocking agents is unexpectedly advantageous and enhances the overall beneficial action of the subject compositions as desensitizing agents. More particularly, combining the copolymers having alternating hydrophilic and hydrophobic monomers, e.g. a long chain α-olefin hydrophobic monomer and a salt-forming carboxylic acid or diacid hydrophilic monomer, with super-absorbent acrylic acid polymers, e.g. polyacrylate polymers, results in a tubule blocking component having enhanced efficacy. The addition of the copolymer to the polyacrylate polymers allows the latter to be present in a lower concentration, yet maintain or even somewhat enhance its relative effectiveness without the previously reported disadvantage of undesirable organoleptic properties.

Advantageously, enhanced properties are realized from combining the polyacrylate polymer blocking agent in a concentration not exceeding about two percent by weight of the composition, preferably from about 0.5 to about 2 percent by weight with the above-described hydrophobic/hydrophilic copolymer in a weight ratio of from about 1:1 to 1:10, preferably from about 1:1 to 1:6, most preferably from about 1:2 to 1:6. This combination is also advantageous in that it is particularly robust or stable in the presence of the inorganic salts of the nerve desensitizing agents, especially the potassium salts, in comparison to other know tubule blocking agents.

More than one type of tubule blocking agent can be used besides those described above, including water-soluble or water-swellable polyelectrolyte partial salt such as polyacrylic acid polymers having molecular weight of 400,000 or more under the tradename of Carpobol® or Noveon®, or cationically charged colloidal particles such as alumina coated silica commercially available under the trademark Nalco®, or soaps such as alkyl phosphates and coco fatty acid soaps (the latter working by a mechanism similar to soap scum formation with endogenous calcium in the oral environment).

The tubule blocking agents are incorporated in the composition in a desensitizing effective amount. This will vary depending on the particular type and form of oral composition and other materials present, including the second essential component of desensitizing agent but most often, the tubule blocking agents will be in an amount of about 0.1 to 15 wt. %, usually from about 0.4 to 10% and most preferably from about 2 to 8 wt. %. Phosphate, coco and alike soaps such as MAPS (Rhodia), may be incorporated to further enhance the physical occlusion. Larger amounts of the tubule blocking agent can also be employed if desired.

The second synergistic component of the subject compositions anti-hypersensitivity agents, also known as nerve desensitizing agents. A variety of such agents are suitable for use in conjunction with the tubule blocking agent for a synergistic effect. The nerve desensitizing agents, which are neurally active with analgesic activity, when trapped in the tubules by the effect of the first component tubule blocking agents, effectively act on the nerve to make the nerve less sensitive, hence reducing the effect on the nerve of any external factors that are able to penetrate the tubules. Any nerve desensitizing agent that is a neuroactive substance, i.e., any ion or salt that has a pain reducing or analgesic activity, is suitable for use in the composition of the present invention.

Examples of suitable nerve desensitizing agents include, without intended limitation, potassium or strontium salts, including potassium bicarbonate, potassium citrate, potassium chloride, potassium nitrate, strontium chloride, strontium acetate, strontium nitrate, and potassium or strontium salts of other similar conjugate acids, and mixtures thereof. In one embodiment, at least one potassium salt and/or at least one strontium salt are used in combination with a source of physiologically acceptable fluoride ion such as stannous fluoride, sodium fluoride, potassium fluoride, mixtures thereof or the like.

As with the first component, the nerve desensitizing agent is incorporated in a desensitizing effective amount. This will vary depending on the particular type and form of oral composition and other materials present, including the first essential component of tubule blocking agent but most often, the nerve agent will be in an amount of about 0.1 to 15 wt. %, usually from about 0.3 to 10% and most preferably from about 2 to 8 wt. %.

The oral compositions of the present invention are typically formulated in the form of tooth pastes or gel dentifrices to be brushed on the teeth, or in the form of mouthwashes. However, other delivery systems may also be used. As non-limiting examples, the desensitizing agent can be formulated into a tooth powder, dentifrice, mouthwash, lozenge, buccal adhesive patch, oral spray, coatings or chewing gum. As these delivery forms are prophetic, higher or lower amounts of these agents may be combined to achieve the desired effect.

The formulations containing the desensitizing combinations of the present invention may contain additional ingredients typically incorporated into oral health care compositions. Suitable ingredients include, without intended limitation, abrasive polishing materials, sudsing agents, flavoring agents, humectants, binders, sweetening agents, and water. Abrasives which may be used in the compositions of the invention include alumina and hydrates thereof, such as alpha alumina trihydrate, magnesium trisilicate, magnesium carbonate, aluminosilicate, such as calcined aluminum silicate and aluminum silicate, calcium carbonate, zirconium silicate, polymethylmethacrylate, powdered polyethylene, silica xerogels, hydrogels and aerogels and the like. Also suitable as abrasive agents are calcium pyrophosphate, insoluble sodium metaphosphate, calcium carbonate, dicalcium orthophosphate, particulate hydroxyapatite and the like. Depending on the form that the oral composition is to take, the abrasive may be present in an amount of from 0 to 70% by weight, preferably 1 to 70% by weight, more preferably from 10 to 70% by weight, particularly when the composition is formulated into a toothpaste.

Humectants contemplated for use in the subject compositions include polyols, such as glycerol, sorbitol, polyethylene glycols, propylene glycol, hydrogenated partially hydrolyzed polysaccharides and the like. The humectants are generally present in amounts of from 0 to 80%, and preferably 5 to 70% by weight for toothpastes. Thickeners suitable for use in the invention, typically silica, may be present at a level from about 0.1 to 20% by weight.

Binders suitable for use in the compositions of the invention include hydroxyethyl cellulose, and hydroxypropyl cellulose, as well as xanthan gums, Iris moss and gum tragacanth. Binders may be present in the amount from 0.01 to 10%. Sweeteners suitable for use, e.g. saccharin, may be present at levels of about 0.1% to 5%.

Fluoride sources commonly used in oral health care compositions, such as sodium fluoride, stannous fluoride, sodium monofluorophosphate, zinc ammonium fluoride, tin ammonium fluoride, calcium fluoride and cobalt ammonium fluoride may be included for delivering anti-caries benefit. Preferred compositions in accordance with the present invention will include a fluoride source. Fluoride ions are typically provided at a level of from 0 to 1500 ppm, preferably 50 to 1500 ppm, although higher levels up to about 3000 ppm may be used.

Surfactants, such as a soap, anionic, nonionic, cationic, amphoteric and/or zwitterionic, may be present within the range of 0 to 15%, preferably 0.1 to 15%, more preferably 0.25 to 10% by weight. Anionic and/or nonionic surfactants are most preferred, such as sodium laulyl sulfate, sodium lauroyl sarcosinate and sodium dodecylbenzene sulfonate. Suitable flavors are usually included in low amounts, such as from 0.01 to about 5% by weight, especially from 0. 1% to 5%.

Compositions in accordance with the present invention may, and preferably will, include antibacterial agents include for example phenolics and salicylamides, and sources of certain metal ions such as zinc, copper, silver and stannous ions, e.g. zinc, copper and stannous chloride, and silver nitrate.

Dyes/colorants suitable for oral health care compositions, i.e. FD & C Blue #1, FD & C Yellow #10, FD & C Red #40, etc., may be employed as well. Various other optional ingredients may be included in the compositions of the invention such as preservatives, vitamins such as vitamin C and E, other anti-plaque agents such as stannous salts, copper salts, strontium salts and magnesium salts. Also included may be pH adjusting agents; anti-caries agents such as calcium glycerophosphate, sodium trimetaphosphate; anti-staining compounds such as silicone polymers, plant extracts and mixtures thereof. Additionally, polymers, particularly anionic polymers, such as polycarboxylates or polysulfonates, or polymers containing both a carboxylate and a sulfonate moiety, phosphonate polymers or polyphosphates may be included.

The various substances mentioned above are conventional ingredients suitable for oral care compositions, e.g., toothpastes, gels, mouthwashes, gums, powders, etc. Except where otherwise noted, references to toothpastes are to be construed as applying to gels as well. Mouthwash forms, mouthwashes and similar preparations may be formulated as well. Such preparations typically comprise a water/alcohol solution, including a flavor component, humectant, sweetener, sudsing agent, and colorant. Mouthwashes can include ethanol at a level of from 0 to 60%, preferably from 5 to 30% by weight.

The compositions in accordance with the present invention containing the subject advantageous desensitizing combinations can be prepared by conventional methods of making oral health care formulations, typically combining the various ingredients in a manner and order recognized in the art of compounding such formulations as being convenient to achieve the desired effects. For example, a gel may be formulated by combining a suitable gelling agent with water to form the gel, combining the remaining ingredients therein and finally making whatever adjustments in volume, appearance and the like as would be conventionally carried out to achieve an elegant dentifrice preparation.

In accordance with the present invention, compositions containing two preferred tubule blocking agents are advantageously prepared by separately neutralizing them with heat in the presence of a base to form premixes, adding the cross-linked acrylic polymer premix to the premix containing the hydrolysis product of an alternating copolymer of a long-chain α-olefin and maleic anhydride premix and then adding the remaining ingredients, including the nerve desensitizing agent, for the desired preparation in a conventional manner. Alternately, the alternating copolymer is neutralized as above with an excess of base and then the acrylic polymer is added thereto under high sheer, thereby neutralizing it with the excess base. It has been found that other methods of combining these ingredients do not provide the same level of stability and activity. As a non-limiting example of forming a composition in accordance with the present invention, the combined tubule blocking agents formed by either method described above are mixed with the nerve desensitizing agent, i.e. potassium nitrate, sodium fluoride, sodium saccharin and non-ionic detergent for 10 minutes at temperature of about 50° C. or higher. Under further cooling (at about 25° C. or lower), glycerin and gums are mixed in for 20 minutes. Titanium dioxide, binder and abrasive are blended in for thirty minutes. Detergent and flavor are then blended in, under vacuum, for another 20 minutes.

It has further been found that it is not beneficial to store the neutralized premixes for an appreciable period of time before combining them into a dentrifice preparation. While the exact mechanism for the loss of efficacy is not known with certainty, it has been found that dentrifice preparations prepared from premixes stored for 24 hours suffer about a 25% loss in efficacy. An even greater loss in efficacy results from combining the two tubule blocking agent, simultaneously neutralizing both with base and heat, and then adding the remaining ingredients in a conventional manner.

Although others skilled in the art may find alternative means of preparation to affect an efficacious composition as described herein, the oral compositions of the present invention are preferably prepared as described above. Even where there is but a single tubule blocking agent, it is preferred to neutralize it with a base and heat and then combine it with the remaining ingredients for the desired dentrifice in a conventional manner. In dentifrice form, the composition may be packaged in a conventional plastic laminate, metal tube or dispenser, or be packaged in separate phases to enhanced appearance. It may be applied to dental surfaces by any physical means, such as a toothbrush, fingertip or by an applicator directly to the sensitive area. Mouthwashes according to the present invention may be presented for use as a conventional gargle or may be packaged in a suitable delivery vehicle for use as a mouth spray. Solid dosage forms examples include pastilles, lozenges, chewing gums, tablets, mouthstrips, balms and the like.

The instant invention will be further described with reference to the following non-limiting examples.

### EXAMPLE 1

Dentifrice formulations were prepared using the ingredients set forth in Table 1. The composition designated Example 209 employs a polyacrylate polymer as a tubule blocking agent in combination with potassium nitrate. The composition of Example 276 employs both polyacrylate and polyanhydride copolymer with potassium chloride, whereas that of Example 271 employs potassium nitrate with the combination of both tubule blockers, i.e. the polyanhydride copolymer (PA 18) and the polyacrylate polymer. Additionally, one placebo example identical to Example 271 was prepared without any tubule blockers with the rest of the ingredients being adjusted proportionally to add up to 100%. Examples 209 and 212 contain comparatively low concentrations of the polyacrylate tubule blocking agent, but are organoleptically acceptable. The formulation of the various Examples is shown in Table 1.

TABLE 1

| | Example | Example | Example | Example | Example |
|---|---|---|---|---|---|
| Ingredients in wt. % | #276 | #271 | #295 | 212 | 209 |
| Polyacrylic acid (Carbopol ® | 0.5 | 0.5 | 0.5 | 2 | 1 |
| 980) | | | | | |
| Polyanhydride Copolymer | 3 | 3 | 3 | 0 | 0 |
| (PA-18 ®) | | | | | |
| Potassium Chloride | 3.8 | 0 | 0 | 0 | 0 |
| Potassium Nitrate | 0 | 5.0 | 5.0 | 5.0 | 5.0 |
| Titanium Dioxide, FD&C | 0.5 | ˜0.5 | ˜0.5 | ˜0.5 | ˜0.5 |
| Blue#1, and D&C Yellow#10 | | | | | |
| Triclosan | 0.3 | 0 | 0 | 0 | 0 |
| Sodium fluoride | 0.31 | 0.25 | 0.25 | 0.24 | 0.24 |
| Hydrated silica | 22.0 | 21.0 | 22.0 | 17 | 18.0 |
| Glycerin | 8 | 8 | 8 | 12 | 20 |
| Sorbitol | 22 | 30 | 30 | 38 | 30 |
| Xanthan gum | 0.5 | .2 | 0.25 | 0.7 | 0.5 |
| Carboxy methylcellulose | 0.5 | .2 | 0.25 | 0 | 0.5 |
| Flavor | 1.0 | 1 | 1 | 1 | 1.0 |
| Fumed silica | 1 | 0 | 0 | 0 | 0 |
| Methyl paraben | 0.1 | 0 | 0 | 0 | 0 |
| Sodium saccharin | 0.3 | 0.3 | 0.3 | 0.3 | 0.3 |
| Poloxamer 407 (Pluronic ® F- | 1.0 | 1 | 1 | 0 | 1.0 |
| 127) | | | | | |
| Sodium lauroyl sarcosinate | 0.6 | 1.1 | 0 | 0 | 0.6 |
| (Hamposyl ® L95) | | | | | |
| Alkyl phosphate soap 40% aq | 0 | 0 | 2.5 | 0 | 0 |
| (MAPS, Rhodia) | | | | | |
| Sodium lauryl sulfate | 0 | 0 | 0 | 1.5 | 0 |
| Sodium hydroxide | 0 | 0 | 0 | 0.2 | 0.3 |
| Potassium hydroxide | 1.7 | 1.2 | 1.7 | 0 | 0 |
| Water | q.s. 100 | q.s. 100 | q.s. 100 | q.s. 100 | q.s. 100 |

The formulations were tested for synergistic/desensitizing using the In vitro 10 method described by Pashley, (J. Periodontology, vol. 55, no. 9, p. 522, September 1994), which is also described in U.S. Pat. No. 5,270,031. In this method, intact human molars free from caries and restorations are sectioned perpendicularly to the long axis of the tooth with a metallurgical saw to form thin sections, or discs, from about 0.4 to about 0.6 mm thick. Sections containing dentin and free of enamel are selected for testing and are then etched with an ethylenediamine tetraacetic acid solution to remove the smear layer. Each disc is mounted into the split chambered device described by Pashley which is a special leak-proof chamber connected to a pressurized fluid reservoir containing a tissue culture fluid. By using a mixture of pressurized nitrogen and carbon dioxide gas, the fluid can be made at physiological pH. To further ensure accuracy, the discs are wetted with human saliva to approximate intra-oral conditions. The apparatus includes a gas capillary tube mounted on a ruler or other measuring instrument. An air bubble is injected into the glass capillary tube. By measuring the displacement of the bubble as a function of time, fluid flow through the dentin disc can be measured.

Following measurement of the baseline fluid flow in the dentin disc, an experimental dentifrice is applied to the external surface with a nylon brush. After a defined period of brushing, the experimental material is rinsed off, and the post-application hydraulic conductance is measured. In this fashion, the ability of various experimental materials both alone and as components of dentifrice systems can be tested for the ability to obstruct fluid flow in the dentinal tubules. The percent flow reduction induced by brushing with the experimental materials using the dentifrice formulations shown in the Examples shown in Table 1 are presented in Table 2.

TABLE 2
Percent Flow Reduction of Examples

| Formulation | After Application |
|---|---|
| Example #276 | −92 |
| Example #271 | −92 |
| Placebo similar to example #271 (no tubule | −33 |
| blocking agents) | |
| Family toothpaste (Colgate Winterfresh Gel - | −41 |
| with no potassium salt) | |
| Example 212 | −82 |
| Example 295 | −98 |
| Example 209 | −57 |

In clinical studies, it was found that formulations combining a tubule blocking agents and a nerve desensitizing agent produced significantly better in vivo results than formulations with only nerve desensitizing agent and no tubule blocking agents.

The action of outward dentin fluid flow in opposing the inward diffusion of solutes through dentin has been reported by Pashley and Matthews (Archs. Oral Biol., Vol. 38, Page 577, 1993). It was experimentally found by testing the tubule occluding components of the present invention that they facilitate inward diffusion of known nerve-acting desensitizing agents, such as potassium nitrate and potassium chloride, through dentin disks against the physiologically relevant outward fluid flow.

Potassium diffusion through human dentin disks was tested under conditions with and without outward fluid flow. The trans-dentinal flow in these experiments was created by a pressure gradient of 15 cm water which approximates the tissue pressure inside the tooth. In the presence of outward (opposing diffusion) flow, the inward diffusion of potassium ions through untreated dentin disks from 500 mM potassium was 90% less than the potassium diffusion without opposing flow. This finding was also made on disks treated with a conventional desensitizing dentifrice containing potassium ions. These results are in contrast to disks treated with the experimental formulations where, in the presence of outward flow, the diffusion of potassium is only reduced by 50% with respect to the no-flow condition. Thus, the delivery of nerve agents through dentin is greatly enhanced by treatment with the experimental dentifrice over untreated dentin, and dentin treated with a conventional desensitizing dentifrice. The formulations used in the diffusion tests are shown in Table 3.

TABLE 3

| | Comparative | |
|---|---|---|
| Ingredients % w/w | Example | Example 277 |
| Sorbitol | 39.0 | 30.0 |
| Precipitated silica | 10.0 | 10.0 |
| Amorphous silica | 12.0 | 12.0 |
| Glycerin | 12.0 | 12.0 |
| Potassium nitrate | 5.0 | 5.0 |
| Polyanhydride resin (PA-18) | 0 | 3.0 |
| Potassium hydroxide | 0 | 1.5 |
| Sodium lauryl sulfate | 1.5 | 0 |
| Poloxamer 407 | 0 | 1.0 |
| Flavor | 1.0 | 1.0 |
| Sodium lauroyl sarcosinate (hamposyl L-95) | 0 | 1.0 |
| Polyacrylic acid (Carbopol ® 980) | 0 | 0.5 |
| Titanium dioxide, FD&C blue #1, and D&C | ˜0.5 | ˜0.5 |
| yellow #10 | | |
| Sodium saccharin | 0.3 | 0.3 |
| Trisodium phosphate | 0.25 | 0 |
| Carboxymethylcellulose | 0 | 0.25 |
| Xanthan gum | 0.7 | 0.25 |
| Sodium fluoride | 0.243 | 0.243 |
| Water | q.s. 100 | q.s. 100 |

### EXAMPLE 2

Formulations including those containing a combination of tubule blocking agents in accordance with the present invention with a desensitizing agent were tested as described in Example 1 for hydraulic conductance. Only one desensitizing agent was utilitzed in each formulation. The results are given in Table 4.

TABLE 4

| | | | | Percent | Mean Post |
|---|---|---|---|---|---|
| | | | Percent w/w | w/w | Application |
| Sample | KNO³ | KCl | Carbopol ® 980 | PA-18 | (ΔLp1) |
| 1 | X | | 1 | 0 | −57^{[n=3]} |
| 2 | X | | 2 | 0 | −82^{[n=2]} |
| 3 | X | | 0 | 2 | −44^{[n=3]} |
| 4 | X | | 1.5 | 2 | −67^{[n=32} |
| 5 | X | | 2 | 2 | −85^{[n=2]} |
| 6 | | X | 0.5 | 2.7 | −80^{[n=2]} |
| 7 | X | | 0.5 | 2.7 | −74^{[n=1]} |
| 8 | X | | 0.9 | 2.7 | −84^{[n=4]} |
| 9 | X | | 0 | 3 | −75^{[n=4]} |
| 10 | X | | 0.5 | 3 | −93^{[n=16]} |
| 11 | | X | 0.5 | 3 | −83^{[n=10]} |
| 12 | X | | 1.5 | 3 | −96^{[n=4]} |
| 13 | | X | 0 | 3.5 | −81^{[n=4]} |
| 14 | X | | 0.5 | 4 | −39^{[n=2]} |

It can be seen from the results given in Table 4 that the combination of two tubule blocking agents in accordance with the present invention, further in combination with a desensitizing agent, gave significantly enhanced results. It can further be seen that the combination of the two tubule blocking agents is superior to increasing the quantity of either one tested individually. As a practical matter, concentrations of Carbopol® 980 in excess of two percent by weight were not tested due to undesirable organoleptic properties. However, the combination of 1.5 percent by weight Carbopol® 980 and 3 percent by weight of PA-18 was found by a taste test panel to be organoleptically acceptable. The tested formulations were shown to be stable in the presence of the potassium salts tested. The results in Table 4 clearly demonstrate the enhanced effects realized by the compositions of the present invention.

### EXAMPLE 3

A mouthwash in accordance with the invention may be made by mixing the ingredients given below

| Ingredient | Percent by Weight |
|---|---|
| Water | 70 |
| Dispersion of DP6-6984 (Crosslinked polyacrylate) | 5 |
| Potassium Nitrate | 3 |
| Alcohol 109 Proof (Grain Alcohol) | 10 |
| Poloxamer 407 (Pluronic ® F-127) | 2 |
| Flavor | 0.2 |
| Menthol | 0.02 |
| Water | q.s. to 100 |

### EXAMPLE 4

A chewing gum in accordance with the invention may be made using the formulation below. The chewing gum base will be softened at 65° C. using a sigma blade mixer, cooled to 60° C. and ⅗ of the sorbitol powder and calcium saccharin will then be added, followed by the glycerin. Then ⅕ of the sorbitol powder, ½ of the lecithin and the superabsorbent polymer will be added. After cooling to 50° C., the rest of the sorbitol powder, lecithin, and flavor will be added. The mixture may then be rolled into patties and cut into strips.

| Ingredient | Percent by Weight |
|---|---|
| Chewing Gum NOVA Base “A” | 24.64 |
| Glycerin | 1 |
| Calcium Saccharin | 0.06 |
| Sorbitol Powder | 53.5 |
| Lycasin | 13 |
| Lecithin | 0.8 |
| Flavor | 1 |
| DP6-6983 (Cross-linked polyacrylate) | 3 |
| Potassium Nitrate | 3 |

### EXAMPLE 5

A lozenge in accordance with the invention may be made using the formulation below. The sorbitol and xylitol are heated at 165° C. until the base begins to thicken. The combination is cooled to 140° C. and the citric acid added. After cooling to 100° C., the gelatin is added and after cooling to 50° C., the flavor and superabsorbent polymer are added. Cooling is continued and a seed crystal of sorbitol is added to start crystallization. The mixture is then poured into molds to form lozenges.

| Ingredient | Percent by Weight |
|---|---|
| Sorbitol | 81.5 |
| Xylitol | 6 |
| Citric Acid | 0.4 |
| Sodium Hydroxide | 0.5 |
| Polyanhydride Resin | 2 |
| Flavor | 0.1 |
| Gelatin | 7 |
| Polyacrylate | 2 |
| Potassium Nitrate | 3 |

1. A dental composition for reducing dentinal hypersensitivity due to the presence of exposed dentinal surface and open dentinal tubules, comprising a suitable carrier and a synergistic combination of: a desensitizing amount of at least one tubule blocking agent selected from an alkyl phosphate or fatty acid soap; and a nerve desensitizing agent selected from the group consisting of at least one potassium salt, at least one strontium salt, and mixtures thereof.

2. A composition in accordance with claim 1, wherein said nerve desensitizing agent is selected from the group consisting of potassium bicarbonate, potassium citrate, potassium chloride, potassium nitrate, strontium chloride, strontium acetate, strontium nitrate, and potassium or strontium salts of other similar conjugate acids, and mixtures thereof.

3-7. (canceled)

8. A composition in accordance with claim 1, wherein said nerve desensitizing agent is at least one potassium salt.

9. A composition in accordance with claim 1, wherein said at least one tubule blocking agent is present in an amount of from 0.1 weight percent to 15 weight percent.

10. A composition in accordance with claim 1, additionally including a source of physiologically acceptable fluoride ion.

11. A composition in accordance with claim 10, wherein said source of physiologically acceptable fluoride ion is selected from the group consisting of stannous fluoride, sodium fluoride, potassium fluoride and mixtures thereof.

12. A method for reducing dentinal hypersensitivity in a sensitive tooth due to the presence of exposed dentinal surface and open dentinal tubules, comprising the step of administering to said sensitive tooth a dental composition comprising a suitable carrier and a synergistic combination of: a desensitizing amount of at least one tubule blocking agent said at least one tubule blocking agent comprising in combination an selected from an alkyl phosphate or fatty acid soap; and a nerve desensitizing agent selected from the group consisting of at least one potassium salt, at least one strontium salt, and mixtures thereof, thereby causing said at least one tubule blocking agent to deposit upon the exposed dentinal surface and precipitate within the dentinal tubules of said sensitive tooth, thereby creating a high concentration of said nerve desensitizing agent at the exposed dentinal surface and within the dentinal tubules.

13. A method in accordance with claim 12, wherein said nerve desensitizing agent is selected from the group consisting of potassium bicarbonate, potassium citrate, potassium chloride, potassium nitrate, strontium chloride, strontium acetate, strontium nitrate, and potassium or strontium salts of other similar conjugate acids, and mixtures thereof.

14-17. (canceled)

18. A method in accordance with claim 12, wherein said nerve desensitizing agent is at least one potassium salt.

19. A method in accordance with claim 12, wherein said composition additionally includes a source of physiologically acceptable fluoride ion.

20. A method for preparing a dentifrice composition for reducing dentinal hypersensitivity due to the presence of exposed dentinal surface and open dentinal, comprising a synergistic combination of: i) a desensitizing amount of at least two tubule blocking agents, a hydrolysis product of an alternating long chain α-olefin-co-maleic anhydride copolymer as the first blocking agent and an acrylic polymer as the second blocking agent; and ii) a nerve desensitizing agent selected from the group consisting of potassium salt, strontium salt, and mixtures thereof, said method comprising:
a) separately neutralizing the tubule blocking agents by heating them in the presence of a suitable base to form premixes;
b) combining the two premixes; and
c) forming said dentifrice composition by mixing the combined premixes with said nerve desensitizing agent and conventional dentifrice ingredients.

21. A method for preparing a dentifrice composition for reducing dentinal hypersensitivity due to the presence of exposed dentinal surface and open dentinal tubules, comprising a synergistic combination of: i) a desensitizing amount of at least two tubule blocking agents, a hydrolysis product of an alternating long chain α-olefin-co-maleic anhydride copolymer as the first blocking agent and an acrylic polymer as the second blocking agent; and ii) a nerve desensitizing agent selected from the group consisting of potassium salt, strontium salt, and mixtures thereof, said method comprising:
a) neutralizing said copolymer in an excess of base;
b) adding said acrylic polymer there under high sheer, thereby neutralizing it with the excess base; and
c) forming said dentifrice composition by mixing the resultant mixture with said nerve desensitizing agent and conventional dentifrice ingredients.
